(19) United States
(12) Patent Application Publication
(10) Pub. No.: US 2008/0221590 A1
(43) Pub. Date: Sep. 11, 2008

(54) APPARATUS FOR POSITIONING AND HOLDING IN PLACE A MANUALLY MANIPULATED MEDICAL DEVICE DURING THE PERFORMANCE OF A ROBOTICALLY ASSISTED MEDICAL PROCEDURE

(75) Inventors: Michael Ikeda, San Jose, CA (US); Scott E Manzo, Shelton, CT (US); Gresham Richard, Guilford, CT (US); George T. Wong, Chelmsford, MA (US); Paul A. Licari, Boxborough, MA (US); Shannon Lee Kelley, Millbrae, CA (US)

Correspondence Address: PATENT DEPT;INTUITIVE SURGICAL, INC, 1266 KIFER RD, BUILDING 101, SUNNYVALE, CA, 94086, US

(73) Assignee: Intuitive Surgical, Inc., Sunnyvale, CA (US)

(21) Appl. No.: 11/847,154

(22) Filed: Aug. 29, 2007

Related U.S. Application Data
(60) Provisional application No. 60/892,951, filed on Mar. 5, 2007.

(51) Int. Cl. A61B 19/00

(52) U.S. Cl. 606/130

(57) ABSTRACT

An apparatus includes two clamp assemblies on opposing sides of an operating table. A cross bar is attached to the assemblies and extends across a lowered leg section of the table. A pivot joint is coupled to the cross bar, a first linkage couples the pivot joint to a first ball joint, a second linkage couples the first ball joint to a second ball joint, and a medical device is coupled to the second ball joint through an adapter. Each joint has a spring mechanism resisting its movement and a hydraulic cylinder to allow its movement. Depression of a foot pedal actuates all hydraulic cylinders so that an operator may position the medical device by moving the pivot joint and attached first linkage rotationally about and directionally along the cross bar, the second linkage rotationally about the first ball joint, and the medical device rotationally about the second ball joint.

### CROSS REFERENCE TO RELATED APPLICATIONS

This application claims priority to U.S. Provisional Application No. 60/892,951 filed Mar. 5, 2007, which is incorporated herein by reference.

### FIELD OF THE INVENTION

The present invention generally relates to medical robotic systems and in particular, to an apparatus for positioning and holding in place a manually manipulated medical device during the performance of a robotically assisted medical procedure.

### BACKGROUND OF THE INVENTION

Medical robotic systems such as those used in performing minimally invasive surgical procedures offer many benefits over traditional open surgery techniques, including less pain, shorter hospital stays, quicker return to normal activities, minimal scarring, reduced recovery time, and less injury to tissue. Consequently, demand for minimally invasive surgery using such medical robotic systems is strong and growing.

Examples of medical robotic systems include the da Vinci® Surgical System and the da Vinci®S™ Surgical System from Intuitive Surgical, Inc., of Sunnyvale, Calif. Each of these systems includes a surgeon's console, a patient-side cart, a high performance three-dimensional (“3-D”) vision system, and Intuitive Surgical's proprietary EndoWrist® articulating instruments, which are modeled after the human wrist so that when added to the motions of manipulators holding the surgical instruments, they allow at least six degrees of freedom of motion, which is comparable to or even greater than the natural motions of open surgery.

The da Vinci® surgeon's console has a high-resolution stereoscopic video display with two progressive scan cathode ray tubes (“CRTs”). The system offers higher fidelity than polarization, shutter eyeglass, or other techniques. Each eye views a separate CRT presenting the left or right eye perspective, through an objective lens and a series of mirrors. The surgeon sits comfortably and looks into this display throughout surgery, making it an ideal place for the surgeon to display and manipulate 3-D intraoperative imagery.

The patient-side cart typically includes three or more robotic arm assemblies with corresponding slave manipulators for holding and manipulating medical devices such as surgical instruments and image capturing devices for performing and/or viewing a medical procedure at a surgical site within a patient. To manipulate these medical devices, the surgeon's console also includes input devices which may be selectively associated with the medical devices and their respective slave manipulators. Since the movements of the input devices and their associated medical devices are scaled, this allows the surgeon to perform intricate medical procedures with greater ease than conventional open surgery. Further, it may even allow the surgeon to perform medical procedures that are not even feasible using conventional open surgery techniques.

To perform a minimally invasive surgical procedure on a patient, one or more incisions are first made in the patient and cannulae inserted therein to gain access to a surgical site within the patient. Setup arms supporting the slave manipulators are then positioned so as to allow the slave manipulators to attach to respective of the cannulae. Surgical instruments engaged on the slave manipulators are then inserted into the cannulae and properly positioned and oriented in order to perform the procedure. A surgeon may then manipulate input devices which are coupled to the slave manipulators and their respective surgical instruments through one or more controllers to perform the surgical procedure.

To perform a gynecological procedure, the patient-side cart may be positioned, as shown in FIG. 1, at one end of an operating table so as to gain better angles of access to the patient's perineum. When the patient-side cart is placed in this position, it may be difficult for an assistant standing on one side of the table to manually position and hold a uterine manipulator or other medical device without undue difficulty or interfering with or being interfered by the movement of the robotic arms of the patient-side cart.

Although an extra robotic arm might be added to the patient-side cart to position and hold the uterine manipulator in place, the cost of the extra arm may be expensive. Therefore, an instrument holder such as the ENDOBOY® arm marketed by Geyser Endobloc, a French company, may be employed instead to position and hold the uterine manipulator. Because the ENDOBOY® arm is clamped to one side of the operating table and has limited range, however, it may be difficult to properly position its held medical device without interfering with or being interfered by the robotic arms of a patient-side cart positioned as shown in FIG. 1. Further, the pneumatic operation of its arm requires use of an external gas source which may not be readily available, and its hand operation of a control lever may be inconvenient for unlocking the arm so that it may be manually positioned.

### OBJECTS AND SUMMARY OF THE INVENTION

Accordingly, one object of one or more aspects of the present invention is an apparatus for positioning and holding in place a manually manipulated medical device during the performance of a robotically assisted medical procedure.

Another object of one or more aspects of the present invention is an apparatus for positioning and holding in place a manually manipulated medical device that does not interfere with and is not interfered by robotic arms of a patient-side cart positioned at a patient's feet or head end of an operating table.

Still another object of one or more aspects of the present invention is an apparatus for positioning and holding in place a manually manipulated medical device that does not require gas tanks or other depletable energy sources for its operation.

These and additional objects are accomplished by the various aspects of the present invention, wherein briefly stated, one aspect is an apparatus for positioning and holding in place a medical device, comprising an arm and an input device. The arm has a first joint assembly capable of rotational movement about and directional movement along an axis extending across a width of an operating table, a first linkage coupled at one end to the first joint assembly so as to move with the first joint assembly, a second joint assembly having a first joint coupled to another end of the first linkage so as to move with the first linkage, a second linkage coupled at one end to the second joint assembly so as to be capable of rotational movement about the first joint of the second joint assembly, and a third joint assembly having a second joint coupled to the second linkage so as to move with the second linkage, wherein the third joint assembly is adapted to hold the medical device so that the medical device is capable of rotational movement about the second joint of the third joint assembly. The input device is coupled to the first, second and third joint assemblies so that the first, second and third joint assemblies allow the first linkage, the second linkage, and the medical device to be moved when the input device is activated.

Another aspect is an apparatus for positioning and holding in place a manually manipulated medical device, comprising: an arm adapted to hold the medical device, the arm including coupled together linkages and joint assemblies, wherein the joint assemblies individually include a joint, a spring mechanism resisting movement of the joint, and an actuator generating a counter force to overcome the spring mechanism and allow movement of the joint when actuated; and an input device coupled to the joint assemblies so as to actuate the actuators when the input device is activated.

Another aspect is an apparatus for positioning and holding in place a manually manipulated medical device, comprising: an arm, a base support, and an input device. The arm is adapted to hold the medical device and includes coupled together linkages and joint assemblies, wherein the joint assemblies individually include a joint, a mechanism resisting movement of the joint, and an actuator generating a counter force to overcome the resistance generated by the mechanism and allow movement of the joint when actuated. The base support includes first and second structures clamped to opposing sides of an operating table, and a cross bar having one end coupled to the first structure and another end coupled to the second structure, wherein the arm is coupled to the cross bar at one end and adapted to hold the medical device at the other end. The input device is coupled to the joint assemblies so as to actuate their actuators when the input device is activated, thereby allowing the medical device to be positioned.

Additional objects, features and advantages of the various aspects of the present invention will become apparent from the following description of its preferred embodiment, which description should be taken in conjunction with the accompanying drawings.

### BRIEF DESCRIPTION OF THE DRAWINGS

FIG. 1 illustrates a perspective view of a patient-side cart positioned at the foot end of an operating table for performing a gynecological procedure.

FIG. 2 illustrates a perspective view of an apparatus for positioning and holding in place a manually manipulated medical device, utilizing aspects of the present invention.

FIG. 3 illustrates a perspective view of a multi-jointed arm used in an apparatus for positioning and holding in place a manually manipulated medical device, utilized in aspects of the present invention.

FIG. 4 illustrates a cut-out view of a ball joint assembly used in an apparatus for positioning and holding in place a manually manipulated medical device, utilized in aspects of the present invention.

### DETAILED DESCRIPTION OF THE PREFERRED EMBODIMENT

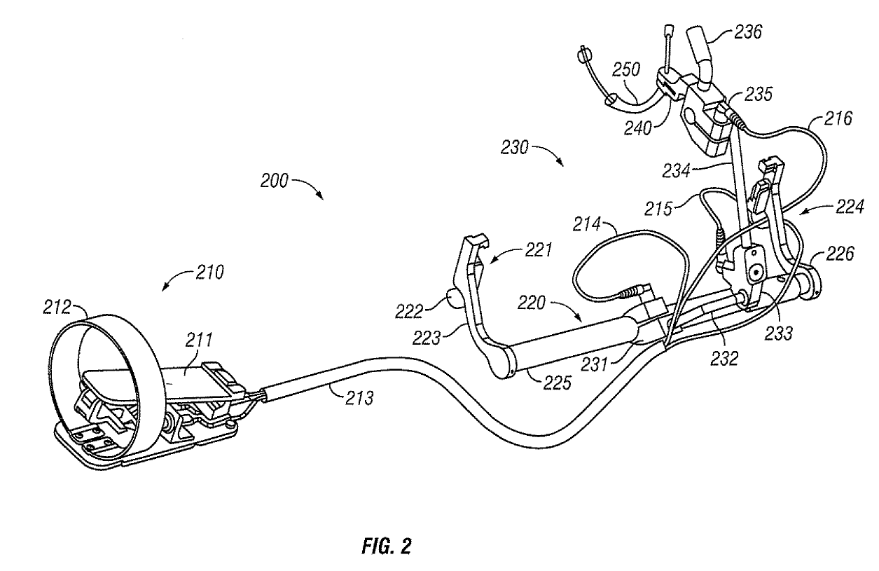

FIG. 2 illustrates, as an example, a perspective view of an apparatus 200 for positioning and holding in place a manually manipulated medical device 250 for gynecological or other medical procedures. An input device 210 includes a foot pedal 211 that when depressed, actuates hydraulic intensifiers (not shown) under the foot pedal 211 to force hydraulic fluid to hydraulic cylinders in joint assemblies 231, 233, 235 through polymer, steel, or carbon fiber reinforced hydraulic lines 214, 215, 216, which are covered by protective tubing 213. A foot guard 212, configured, for example, as a metal or plastic ring, is provided as part of the input device 210 to prevent unintentional depression of the foot pedal 211, such as may occur if it were accidentally kicked over. It also prevents the input device 210 from rolling over, and can additionally be used as a handle for carrying the input device 210.

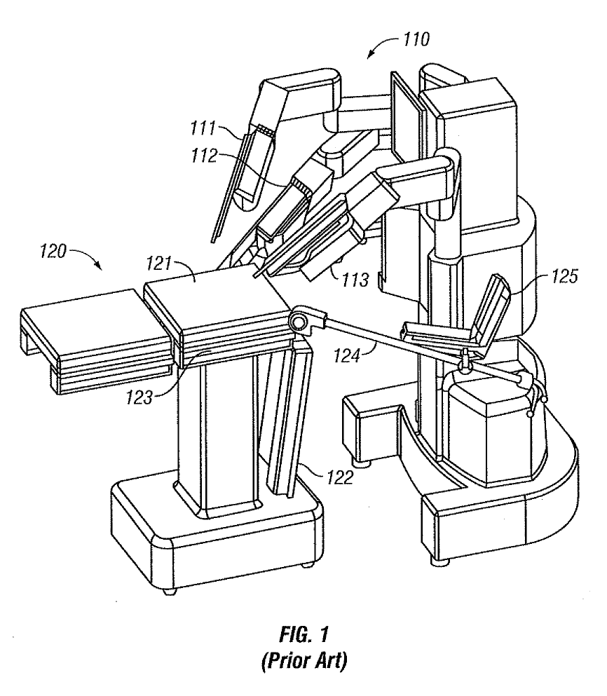

A base support 220 includes a cross bar 225 and clamp assemblies 221, 224. The clamp assemblies 221, 224 respectively attach to opposing side rails of an operating table (such as side rail 123 of the operating table 120 in FIG. 1). The clamp assembly 221 has a downward and outwardly extending structure 223 and a hand actuated (knob driven) clamp mechanism 222, which when actuated, securely clamps the assembly 221 onto the side rail. The clamp assembly 224 is correspondingly constructed.

The cross bar 225 has ends respectively coupled to the downward and outwardly extending structures of the clamp assemblies 221, 224 so that when the clamp assemblies 221, 224 are clamped to their respective side rails of the operating table, the cross bar 225 extends across a width of the operating table and over a lowered leg section of the operating table (such as leg section 122 in FIG. 1). The downward and outwardly extending structures 223, 226 of the clamp assemblies 221, 224 are configured so that their angles and extensions position the cross bar 225 out of the way of rods supporting foot stirrups (such as rod 124 supporting foot stirrup 125 in FIG. 1) and out of the range of movement of robotic arms of a patient-side cart (such as robotic arms 111-113 of patient-side cart 110 in FIG. 1) when performing gynecological or other medical procedures on a patient.

A multi-joint arm 230 includes a pivot joint assembly 231, first and second ball joint assemblies 233, 235, and first and second linkages 232, 234. The pivot joint assembly 231 is coupled to the cross bar 225, the first linkage 232 couples the pivot joint assembly 231 to the first ball joint assembly 233, and the second linkage 234 couples the first ball joint assembly 233 to the second ball joint assembly 235.

An adapter 240 is coupled to the second ball joint assembly 235 to hold a medical device 250, such as a uterine manipulator. Different medical devices may require different adapters. A handle 236 is also coupled to the second ball joint assembly 235 so as to aid a surgical staff member (e.g., a person participating in performing a medical procedure on a patient) in manually moving the multi-joint arm 230 to position the medical device 250 as part of the medical procedure.

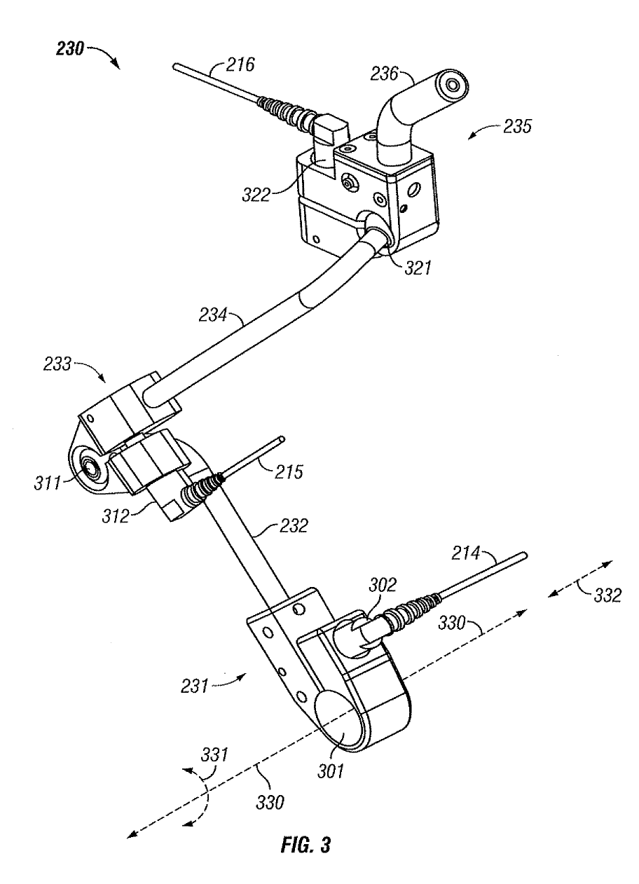

FIG. 3 illustrates, as an example, a perspective view providing more detail of the multi-joint arm 230. The pivot joint assembly 231 includes a pivot joint 301 which may be in the form of, for example, two split halves of a cylinder that are spring loaded so as to apply pressure against the cross bar 225 when it has been inserted therein, and thus inhibit rotational movement about and directional movement along an axis of the cross bar 225. For example, when the cross bar 225 is inserted into the pivot joint 301, its axis is aligned with a central axis 330 of the cylinder formed by joining together the two split halves of the pivot joint 301. Rotational movement about the axis 330 would then be as shown by double headed arrow 331 and directional movement along the axis would be as shown by double headed arrow 332. Also included in the pivot joint assembly 231 is a spring for applying the load or force to normally push the two halves of the cylinder of the pivot joint 301 together, and a hydraulic cylinder 302 connected to the hydraulic line 214 so as to apply a counter force to open or otherwise force apart the two halves of the cylinder of the pivot joint 301 when fluid is forced through the hydraulic line 214 by activation of the input device 210 (e.g., depression of the foot pedal 211).

The linkage 232 is coupled to the pivot joint assembly 231 so that when the pivot joint 301 is prevented from moving relative to the cross bar 225, the linkage 232 is also prevented from moving. Conversely, when the pivot joint 301 is allowed to move rotationally about and directionally along the axis of the cross bar 225, the linkage 232 moves with the pivot joint assembly 231 to move rotationally about and directionally along the axis of the cross bar 225.

The first ball joint assembly 233 includes a ball or other universal joint 311 which allows rotation in all directions when released. Like the pivot joint 301, the ball joint 311 is also spring loaded so that its rotational movement is inhibited. The first ball joint assembly 233 also includes a spring for applying the load or force to inhibit rotation of the ball joint 311, and a hydraulic cylinder 312 connected to the hydraulic line 215 so as to apply a counter force to allow rotational movement of the ball joint 311 when fluid is forced through the hydraulic line 215 by activation of the input device 210 (e.g., depression of the foot pedal 211).

The linkage 232 is coupled to the ball joint 311 so that the ball joint 311 moves with the linkage 232. The linkage 234, on the other hand, is coupled to a joint housing of the first ball joint assembly 233 so that when the joint housing of the first ball joint assembly 233 is prevented from rotating about the ball joint 311, the linkage 234 is also prevented from rotating about the ball joint 311. Conversely, when the joint housing of the first ball joint assembly 233 is allowed to rotate about the ball joint 311, the linkage 234 is also allowed to rotate about the ball joint 311.

The second ball joint assembly 235 also includes a ball or other universal joint 321 which allows rotation in all directions when released. Like the ball joint 311, the ball joint 321 is also spring loaded so that its rotational movement is inhibited. The second ball joint assembly 235 also includes a spring for applying the load or force to inhibit rotation of the ball joint 321, and a hydraulic cylinder 322 connected to the hydraulic line 216 so as to apply a counter force to allow rotational movement of the ball joint 321 when fluid is forced through the hydraulic line 216 by activation of the input device 210 (e.g., depression of the foot pedal 211).

Similar to the coupling of the linkage 232 to the ball joint 311, the linkage 234 is coupled to the ball joint 321 so that the ball joint 321 moves with the linkage 234. The adapter 240 and consequently, the medical device 250, on the other hand, is coupled to a joint housing of the second ball joint assembly 235 so that when the joint housing of the second ball joint assembly 235 is prevented from rotating about the ball joint 321, the adapter 240 and consequently, the medical device 250, is also prevented from rotating about the ball joint 321. Conversely, when the joint housing of the second ball joint assembly 235 is allowed to rotate about the ball joint 321, the adapter 240 and consequently, the medical device 250, is also allowed to rotate about the ball joint 321.

Thus, when a member of the surgical staff grasps the handle 236 and steps on the foot pedal 211 (i.e., activates the input device 210), the staff member may then position and orient a medical device coupled to the second ball joint assembly 235 through an adapter (such as the medical device 250 and adapter 240 in FIG. 2), by manually moving the handle 236 so as to move the pivot joint 301 (and consequently, the first linkage 232) rotationally about and/or directionally along the axis of the cross bar 225, and/or move the second linkage 234 rotationally about the ball joint 311, and/or move the medical device coupled to the second ball joint assembly 235 rotationally about the ball joint 321. Once the medical device is thus positioned and oriented as desired, the staff member may then take his or her foot off of the foot pedal 211 (i.e., deactivate the input device 210), so that the pivot joint 301, the first ball joint 311, and the second ball joint 321 are prevented from moving by their respective spring loaded mechanisms.

Although the input device 210 is described as simultaneously actuating the hydraulic cylinders 302, 312, 322, it is to be appreciated that the input device 210 may alternatively actuate the hydraulic cylinders in a non-simultaneous fashion. For example, the input device 210 may be constructed so that depressing the foot pedal 211 once causes hydraulic fluid to be forced through only hydraulic line 216 to actuate only the hydraulic cylinder 322, depressing the foot pedal 211 a second time causes hydraulic fluid to also be forced through hydraulic line 215 to also actuate the hydraulic cylinder 312, and depressing the foot pedal 211 a third time causes hydraulic fluid to be forced through line 214 to further actuate the third hydraulic cylinder 302. Releasing the foot pedal 211 for more than a period of time, such as three seconds, would then cause hydraulic fluid to no longer be forced through any of the hydraulic lines 216, 215, 214 so that the joint assemblies 231, 233, 235 are locked in place. As another example, the input device 210 may be constructed to also include three buttons, each associated with a different one of the hydraulic cylinders 302, 312, 322. In this case, either the foot pedal 211 could be depressed to actuate all hydraulic cylinders 302, 312, 322 simultaneously, or individual of the buttons may be depressed to only actuate its associated hydraulic cylinder.

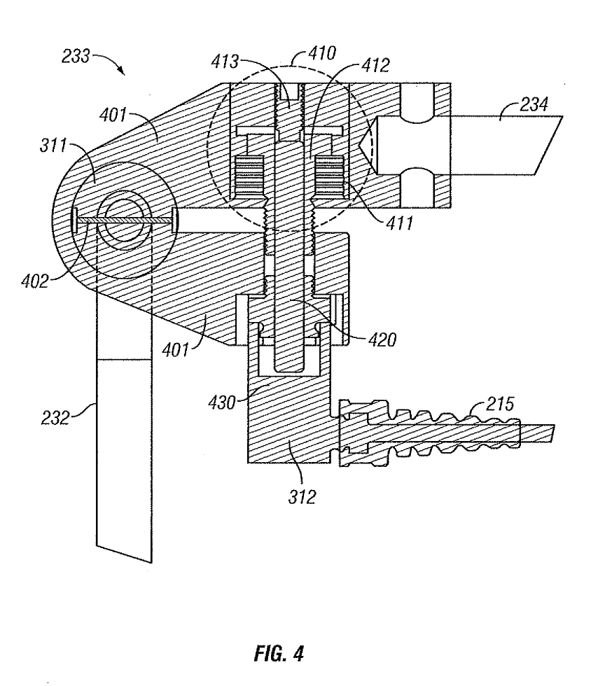

FIG. 4 illustrates, as an example, a cut-out view providing further detail of the ball joint assembly 233. The ball joint 311 is coupled to the linkage 232 by a pin 402 that passes through aligned holes in the ball joint 311 and linkage 232 so that the ball joint 311 is fixed relative to the coupled end of the linkage 232. The linkage 234, on the other hand, is attached to the joint housing 401. Therefore, when the ball joint 311 is free to rotate, the ball joint housing 401 and consequently, the linkage 234 may be rotated about the ball joint 311.

A spring mechanism 410 forces the joint housing 401 that surrounds the ball joint 311 to press against its surface with sufficient force to hold it in place under normal operating conditions (e.g., a sufficient clamping force to hold a 10 pound weight at the adapter 240, but not so large that it can't be readily overpowered by a staff member for emergency purposes). As an example of the spring mechanism 410, a Belleville adjuster 412 so as to preload stacked Belleville washers 411 until a desired spring force is provided by the Belleville washers 411 to force opposing sides of the joint housing 401 to clamp the ball joint 311 in place.

The hydraulic cylinder 312 receives hydraulic fluid through the hydraulic line 215 so that when the input device 210 is activated (e.g., by depressing foot pedal 211), hydraulic fluid is forced through the hydraulic line 215 to force a piston 430 in the hydraulic cylinder 312 to push a dowel pin 420 which is set solid against the adjustment set screw 413. As a result, this creates a counter force working against the spring force of the spring mechanism 410 that allows opposing sides of the joint housing 401 to open enough around the ball joint 311 to allow the joint housing 401 to rotate about the ball joint 311.

As previously described, when the input device 210 is activated (e.g., by depressing its foot pedal 211), a hydraulic pump in the input device 210 pumps hydraulic fluid through all of the hydraulic lines 214, 215, 216 so that hydraulic cylinders 302, 312, 322 in the joint assemblies 231, 233, 235 are actuated and their respective joint housings are moveable with respect to their joints 301, 311, 321 (i.e., released from their locked or spring loaded positions) so that the medical device 250 may be positioned as desired by a surgical staff member. Once the medical device 250 is positioned and oriented as desired, the staff member may then deactivate the input device 210 (e.g., by taking his or her foot off of the foot pedal 211) so that the pumped fluid is forced back by the spring mechanisms of the joint assemblies 231, 233, 235 through hydraulic lines 214, 215, 216 back to the input device 210. Since the hydraulic fluid is recycled in this manner, it is practically never used up (except for possible leakage that may be replenished while performing maintenance on the input device 210). Consequently, unlike pneumatic lock/release mechanisms using gas tanks that need to be periodically replenished or replaced after use, the spring/hydraulic lock/release mechanism used in the apparatus 200 for positioning and holding in place the medical device 250 is self contained and thus, more economical, convenient, and possibly safer, to use.

The pivot and ball joint assemblies 231, 233, 235 each operate in substantially the same manner with respect to their housings, spring loaded mechanisms and hydraulic cylinders. The primary differences between the joint assemblies 231, 233, 235 are the linkages and other structures that they are coupled to (and in the case of the pivot joint assembly 231, how it is coupled to the cross bar 225).

For example, the ball joint 321 of the second ball joint assembly 235 is coupled to the linkage 234 by a pin that passes through aligned holes in the ball joint 321 and linkage 234. Consequently, the ball joint 321 is fixed relative to the coupled end of the linkage 234. The adapter 240 and medical device 250, on the other hand, are attached to the joint housing of the second ball joint assembly 235. Therefore, when the ball joint 321 is free to rotate, the joint housing of the second ball joint assembly 235 and consequently, the adapter 240 and medical device 250, may be rotated about the ball joint 321.

The pivot joint 301 of the pivot joint assembly 231, on the other hand, is coupled to the cross bar 225 by inserting the cross bar 225 into the pivot joint 301 to approximately the center of the cross bar 225. The joint housing of the pivot joint assembly 231 is similarly constructed as the joint housing 401 of the first ball joint assembly 233 so that a spring mechanism (such as the spring mechanism 410 of the first ball joint assembly 233) clamps the cross bar 225 in place in the pivot joint 301. A hydraulic cylinder of the pivot joint assembly 231, which operates like the hydraulic cylinder 312 of the first ball joint assembly 233, allows the joint housing of the pivot joint assembly 231 to move rotationally about and directionally along an axis of the cross bar 225 when the hydraulic cylinder 302 is actuated with hydraulic fluid received through hydraulic line 214 from the input device 210. The linkage 232 is attached to the joint housing of the pivot joint assembly 231 so that it also moves rotational about and directionally along the axis of the cross bar 225 as the joint housing of the pivot joint assembly 231 is so moved.

Although the various aspects of the present invention have been described with respect to a preferred embodiment, it will be understood that the invention is entitled to full protection within the full scope of the appended claims.

We claim:

1. An apparatus for positioning and holding in place a manually manipulated medical device, comprising:
an arm having a first joint assembly capable of rotational movement about and directional movement along an axis extending across a width of an operating table, a first linkage coupled at one end to the first joint assembly so as to move with the first joint assembly, a second joint assembly having a first joint coupled to another end of the first linkage so as to move with the first linkage, a second linkage coupled at one end to the second joint assembly so as to be capable of rotational movement about the first joint of the second joint assembly, and a third joint assembly having a second joint coupled to the second linkage so as to move with the second linkage, wherein the third joint assembly is adapted to hold the medical device so that the medical device is capable of rotational movement about the second joint of the third joint assembly; and
an input device coupled to the first, second and third joint assemblies so that the first, second and third joint assemblies allow the first linkage, the second linkage, and the medical device to be moved when the input device is activated.

2. The apparatus according to claim 1, wherein the first, second and third joint assemblies individually comprise:
a joint;
a joint braking mechanism that resists rotational movement of the joint when the input device is not activated; and
a joint releasing mechanism that allows rotational movement of the joint when the input device is activated.

3. The apparatus according to claim 2, wherein the joint braking mechanism includes a spring mechanism exerting a spring force to resist rotational movement of the joint, and the joint releasing mechanism is an actuator exerting a counter force to overcome the spring force to allow rotational movement of the joint when the actuator is actuated by activation of the input device.

4. The apparatus according to claim 3, wherein the actuator includes a hydraulic cylinder and the input device causes fluid to move a piston of the hydraulic cylinder when the input device is activated.

5. The apparatus according to claim 3, wherein the actuator includes a solenoid and the input device causes the solenoid to move a member when the input device is activated.

6. The apparatus according to claim 3, wherein the input device includes a foot pedal and is activated when the foot pedal is depressed.

7. The apparatus according to claim 6, wherein the input device includes a foot guard for preventing accidental depression of the foot pedal.

8. The apparatus according to claim 1, further comprising:
a base support coupled to an operating room structure;
wherein the arm has a first end adapted to hold the medical device and a second end coupled to the base support.

9. The apparatus according to claim 8, wherein the operating room structure includes an operating room table, and the base support comprises:
a first assembly positionable and clampable to one side of the operating room table;
a second assembly positionable and clampable to another side of the operating room table; and
a cross bar having a first end coupled to the first assembly and a second end coupled to the second assembly.

10. The apparatus according to claim 9, wherein the first assembly includes a first downward extending structure coupled to the first end of the cross bar and the second assembly includes a second downward extending structure coupled to the second end of the cross bar, so that the cross bar is positioned away from and at a lower elevation than a top surface of the operating room table.

11. The apparatus according to claim 9, wherein the first joint assembly couples the first linkage to the cross bar, and the first joint assembly includes a pivot joint that allows the first linkage to move rotationally about and directionally along an axis of the cross bar when the input device is activated.

12. The apparatus according to claim 11, wherein the first joint is a first universal joint that allows the second linkage to rotate about the first universal joint when the input device is activated.

13. The apparatus according to claim 12, wherein the second joint is a second universal joint that allows the medical device to rotate about the second universal joint when the input device is activated.

14. The apparatus according to claim 13, further comprising:
a handle coupled to the third joint assembly to facilitate manual positioning of the medical device when the input device activated.

15. The apparatus according to claim 9, wherein the medical device is a uterine manipulator, the cross bar is positioned above and across a lowered leg section of the operating room table, and the arm is positioned so as to direct the medical device towards a perineum of a patient reclining on the operating room table.

16. An apparatus for positioning and holding in place a manually manipulated medical device, comprising:
an arm adapted to hold the medical device, the arm including coupled together linkages and joint assemblies, wherein the joint assemblies individually include a joint, a spring mechanism resisting movement of the joint, and an actuator generating a counter force to overcome the spring mechanism and allow movement of the joint when actuated; and
an input device coupled to the joint assemblies so as to actuate the actuators when the input device is activated.

17. An apparatus for positioning and holding in place a manually manipulated medical device, comprising:
an arm adapted to hold the medical device, the arm including coupled together linkages and joint assemblies, wherein the joint assemblies individually include a joint, a mechanism resisting movement of the joint, and an actuator generating a counter force to overcome the resistance generated by the mechanism and allow movement of the joint when actuated;
a base support including first and second structures clamped to opposing sides of an operating table, and a cross bar having one end coupled to the first structure and another end coupled to the second structure, wherein the arm is coupled to the cross bar at one end and adapted to hold the medical device at the other end; and
an input device coupled to the joint assemblies so as to actuate the actuators of the joint assemblies when the input device is activated, thereby allowing the medical device to be positioned.
